(19) United States
(12) Patent Application Publication
(10) Pub. No.: US 2024/0164711 A1
(43) Pub. Date: May 23, 2024

(54) METHOD FOR IMPROVING TRADITIONAL CHINESE MEDICINE DIAGNOSIS PROCEDURES AND TREATMENT PLANS

(71) Applicant: Yong Yu, Rochester, MI (US)

(72) Inventor: Yong Yu, Rochester, MI (US)

(21) Appl. No.: 18/057,964

(22) Filed: Nov. 22, 2022

(51) Int. Cl. A61B 5/00; G16H 10/60; G16H 20/90; G16H 50/20

(52) U.S. Cl. CPC A61B 5/4854; G16H 10/60; G16H 20/90; G16H 50/20

(57) ABSTRACT

A method for improving Traditional Chinese Medicine diagnosis procedures and treatment plans, wherein the method embodies a cloud computing and relational databases.

### BACKGROUND OF THE INVENTION

The present invention relates to methods of improving Traditional Chinese Medicine and, more particularly, a method for improving Traditional Chinese Medicine diagnosis and treatment, wherein the method embodies a feedback process performed by cloud-based system, wherein machine learning automatically executes the feedback and modification processes.

All methods of diagnostic procedures in Traditional Chinese Medicine (TCM) use human senses as diagnostic sensors. As such, judgement is more qualitative than quantitative. Due to the subjective nature of qualitative analysis, different TCM practitioners may arrive at different diagnoses or diagnostic severities for the same symptoms in the same patient. Therefore, consistency and effectiveness of treatment of any ailments by using Chinese TCM varies.

The subjectivity of TCM practices also causes treatment methods of each disease to be inconsistent across the TCM community. Furthermore, the inconsistency of diagnoses and treatments means the methods of TCM cannot be standardized nor be statistically evaluated. Because of this lack of standardization, some of the most effective treatment methods may have been lost to history.

Currently, TCM systems available on the market are instruction-based systems that are not receptive to any forms of feedback. This is in part due to these systems not having an intention to use the feedback data; thus, there are no evaluation systems geared towards improving TCM treatments through leveraging statistical evaluation.

As can be seen, there is a need for a method for improving consistency and effectiveness of Traditional Chinese Medicine diagnosis and treatment, wherein the method embodies cloud computing and relational databases to quantitatively improve the treatment plans of TCM through feedback system to modify the relational databases automatically through Machine Learning.

### SUMMARY OF THE INVENTION

In the system of the present invention, the severity of symptoms is quantified as the practitioners input indexed information regarding the patients' conditions. This system is configured to generate diagnoses as well as provide the availability for practitioners to submit diagnoses, overriding or supplementing the system-generated diagnosis. Likewise, the system is configured to suggest TCM treatment methods as well as allow the practitioners to submit treatment methods. The system of the present invention embodies a feedback loop whereby diagnosis and treatment methods (outputs) are assigned accuracy scores (feedback inputs) that are fed back into the system so as to effect future outputs (e.g., future diagnosis and treatment methods). The feedback loop includes diagnoses of ailments, application of TCM treatments (including medicines, acupuncture, cupping, and other methods), observation of the results of treatments, evaluation of the effectiveness of treatments, and a standardized score (the feedback inputs) for effectiveness of each clinical action and diagnosis. Based on the evaluation of data regarding treatment efficacy, the treatment methods of TCM can be improved and standardized across a population. This process will elevate the TCM from individualized empirical acts into practices with statistically significant effectiveness.

This TCM system will generate diagnoses according to the symptoms of the patient provided by TCM practitioners based on all available TCM knowledge. As mentioned above, the system also provides the flexibility for TCM practitioners to generate their own diagnoses. Based on the diagnosis provided by the practitioners, the system will generate treatment methods. TCM practitioners will also have the flexibility to create their own treatment methods. The effectiveness of the treatment will be evaluated by the follow-up visits. By making the platform cloud based, the system will be able to collect data across a wide spectrum of patients. It will then provide the opportunity to improve and standardize the TCM methods. With accumulation of the data, the system can statistically evaluate the effectiveness for each individual and combine TCM methods and medicine against each targeted ailment, and to create statistically supported TCM methods and medicines for treating diseases.

By adding a centralized relational database component to the TCM system, a feedback component, and an efficacy evaluation component, the method will be able to make statistically data backed improvements to TCM, and executable by Machine Learning. Thereby, the system embodied in the present invention elevates TCM into a data supported method with the effect of standardizing TCM practices across the TCM community.

In one aspect of the present invention, a cloud-based Tradition Chinese Medicine (TCM) having systemic hardware and software includes the following: the hardware comprising: servers and data storage equipment at one or multiple cloud computing facilities and multiple client-side electronic devices; the cloud server system comprising of physical hardware modules to accomplish the functional needs of data management, data analysis, processing, and client-server communication; the software of the Traditional Chinese Medicine System resides in the cloud platform, wherein the software is the tool to realize the intended purpose of generating accurate and consistent TCM diagnoses and the effective medical treatment actions, wherein there are several modules including the Request Processing module, the TCM-Knowledge module, the Electronic Health Record (EHR) module, the TCM Expert System module, TCM Evaluation, Rating, and Modification (ERM) module and the database management module which form the main structure of the software; the cloud platform provides the system the capability to collect enough data to statistically evaluate each diagnosis and treatment method; this evaluation is in turn to be used to improve the TCM rules and manuscripts, wherein the Request Processing module receives various types of requests from various types of users and applications. Upon receipt of the requests, it invokes other modules to accomplish the request. The client-side users access the system through an authentication process and the users are assigned different levels of access and request privileges, wherein the TCM-Knowledge module stores existing TCM theories, traditional practices and various types of TCM examples, and the knowledge from available TCM theories, books, and publications. In this module the relationship between a combination of symptoms or physiological characteristics and diagnoses based on existing TCM theories, traditional practices and various types of TCM examples and manuscripts are constructed in the form of relational data. The primary field of the data is structured based on the classification of type of ailments of TCM with reference to the analogous classification of modern medicine. The TCM medicine database is organized based on medicinal functions per definition from TCM, wherein the Electronic Health Record (EHR) module includes various types of applications used by medical service providers, medical insurance organizations, medical service organizations, pharmacies and/or other applications that establish, access and store patients' medical information. The database attached to this module stores social and medication information of each individual target. The EHR module data is stored both locally on client-side devices as well as in the server-side data storage facilities, wherein the TCM expert system module provides diagnoses and medical treatment instructions for individual patients. The initial TCM knowledge in the TCM expert system is directly transcribed from the information in the TCM-Knowledge center module. The information associated with making diagnoses such as medical information, physiological and psychological information and clinical actions that apply to each patient are synchronized with EHR. The relationship between diagnoses and symptoms and other descriptions and the combination of symptoms and other descriptions are built in the optimized TCM rules. Treatment instructions are also built in the optimized TCM rules. The practitioners have the liberty to adopt any subset of the diagnoses and treatment instructions generated by the TCM system as well as the option to submit their own diagnoses and treatment plans. The database attached to this module stores the optimized TCM rules, wherein the TCM Evaluation, Rating, and Modification Module (ERM) generates statistical evaluations of all TCM treatment methods and practitioners. With the aggregate patient information collected in the system, statistical evaluations of all TCM diagnoses and treatment methods and practitioners are generated. The TCM rules are iterated and optimized based on the evaluation of the result generated in this module. The database of ERM module extracts data from EHR to enable the module to generate a score to rate the accuracy of the diagnosis and a score to rate the efficacy of the treatment method based on the improvement of the patient's health, the cloud based TCM system, wherein the Database Management module manages the data of the whole system. The database of all modules in the system forms a network-like data structure.

In another aspect of the present invention, a method for cloud TCM system comprising: the data of patients and other requests are input via client-side devices. The data of patients is stored into client EHR system and synchronized with the TCM database; requests are transmitted to the cloud system through a secure communications medium; upon receipt of the input data and the requests, the cloud TCM system authenticates the requests and analyzes the data input and requests; using data from the TCM knowledge, TCM medicine, and TCM expert data, the TCM expert module generates a diagnosis and treatment plan in response to the requests, wherein the system then sends the diagnosis and the medical action instructions back to client terminal; and the TCM practitioner at the client terminal has the option to accept or modify the system generated treatment plan or submit their own recommended treatment plan on each patient visit, and wherein a subsequent visits from the patients, the results of treatments from previous visits are input into the system. Based on the results, an effectiveness score of prior treatments to the specific ailment is generated by the system to form a closed loop process. Any deviation of treatment from existing TCM rules is evaluated by the system. If a new treatment method is more effective than an existing one, the recommended treatment methods will be modified to reflect the improvement. The system will be constantly iterated to adopt the most effective TCM methods.

In yet another aspect of the present invention, a method for cloud the TCM system above wherein the TCM knowledge including TCM theories, traditional practices, and various types of TCM examples are input or uploaded into the system at its inception, wherein the TCM medicine catalog is input or uploaded into the system at the initiation of the system, wherein the initial data of the TCM expert system is a direct copy of that of TCM knowledge, wherein the TCM rules in the database of TCM Expert system can be modified and updated, and wherein the cloud ERM module generates a score rating the accuracy of the diagnosis and a score rating the efficacy of the treatment method based on the improvements to the patient's health.

In another aspect of the present invention, the system for improving Traditional Chinese Medicine (TCM) including the following: a processor, and a memory comprising computing device-executable instructions that, when executed by the processor, cause the processor to implement: a communications interface for accessing a TCM collaboration platform over a network; a user interface for displaying and interacting with the TCM collaboration platform; a TCM-Expert (TCME) module retrievably storing a plurality of TCM parameters, wherein the TCME module is configured to generate one or more of the plurality of TCM parameters upon request, wherein each TCM parameter comprises a TCM diagnosis and at least one TCM clinical action; an Electronic Health Record (EHR) module retrievably social and medication information of each individual target; and a TCM Evaluation, Rating, and Modification (ERM) module generates statistical evaluations of said TCM parameters and TCM practitioners, wherein the ERM module extracts data from the EHR module to generate an efficacy score of each TCM parameter, wherein an amplitude of each efficacy score is based on improvement of the respective individual target, and wherein the efficacy score is fed back into the TCME module for the associated TCM parameter, wherein the ERM module will generate an accuracy score for each TCM diagnosis and TCM clinical action, respectively, based on the associated efficacy score, further including a machine learning module configured to process rules to modify the TCME module by training the machine learning module, comprising repeatedly: receiving an efficacy score; accessing the TCM parameter corresponding at least in part to the efficacy score; and identifying and extracting one or more TCM parameter stored in the TCME module based on said efficacy score, wherein the machine learning module replaces the extracted TCM parameter with another TCM parameter having an efficacy score of a higher amplitude, whereby improving the machine learning module accuracy in identifying TCM parameter deficiencies and improving the system.

In another embodiment, the system includes wherein the machine learning module is configured to process rules to identifying and extracting one or more TCM diagnosis and TCM clinical action from each TCM parameter stored the TCME module based on each respective accuracy score, wherein the machine learning module replaces the extracted one or more TCM diagnosis and TCM clinical action with another one or more TCM diagnosis and TCM clinical action having an accuracy score of a higher amplitude, further including a database management module configured to manage the data of the TCM collaboration platform to form a network-like data structure, wherein the network is a cloud-based network.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention.

FIG. 2 is a diagrammatical view of an exemplary embodiment of an operating system of the present invention.

FIG. 3 is a diagrammatical view of an exemplary embodiment of an input request processing module of the present invention.

FIG. 4 is a flow chart of an exemplary embodiment of the present invention.

FIG. 5 is a flow chart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a cloud system and a method for improving Traditional Chinese Medicine (TCM) diagnosis procedures and treatment plans, wherein the method embodies a feedback process executable automatically by Machine Learning that self-modifies the databases of the system in real time.

The system described in the present invention includes a TCM expert system module which provides diagnostics and medical instruction to treat the patients. The TCM evaluation, rating, and modification module generates an effectiveness score for each clinical action for each disease. The database center collects all medical actions and treatment results from every TCM practitioner connected to the system. The results of clinical actions are further used for evaluation and optimization of TCM best practices. With accumulation of data, the system can statistically evaluate the effectiveness for each individual and combine TCM methods and medicine against each targeted ailment, and to create statistically supported TCM methods and medicines for treating diseases.

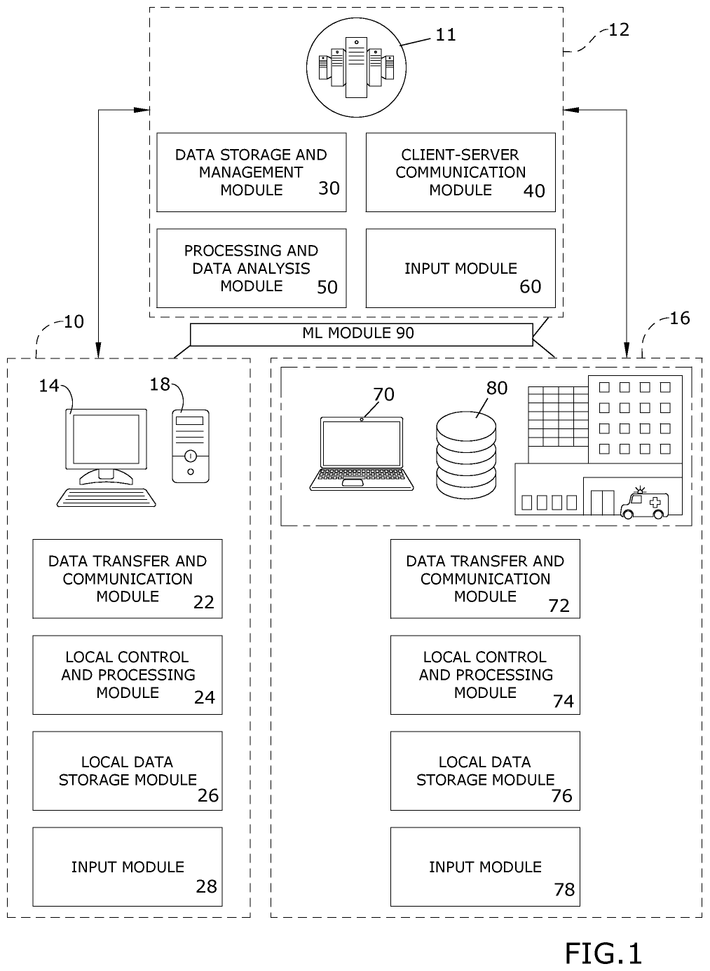

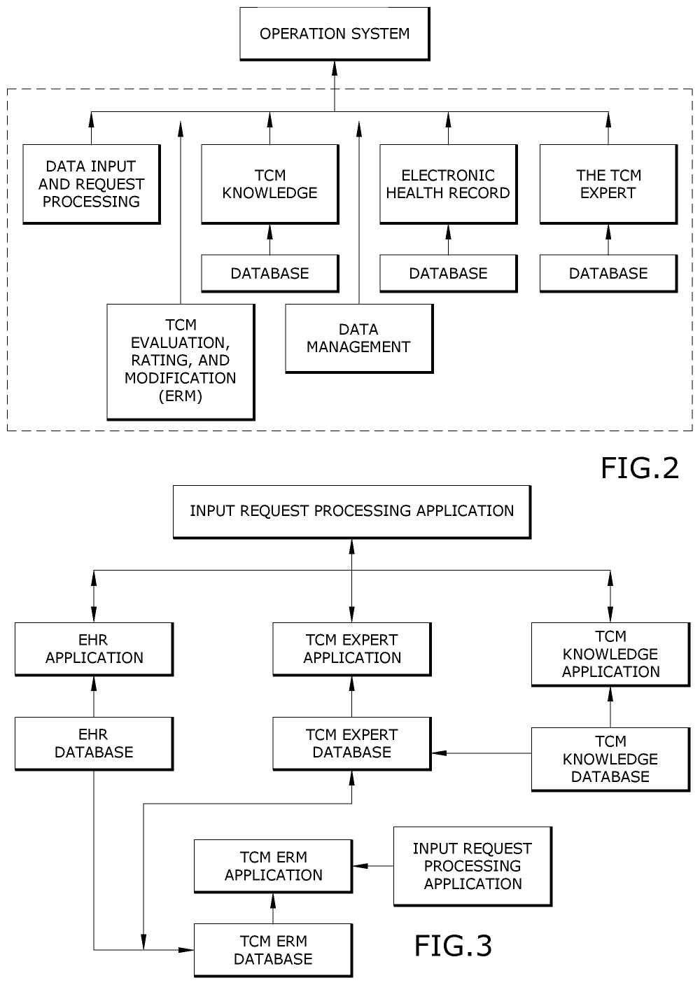

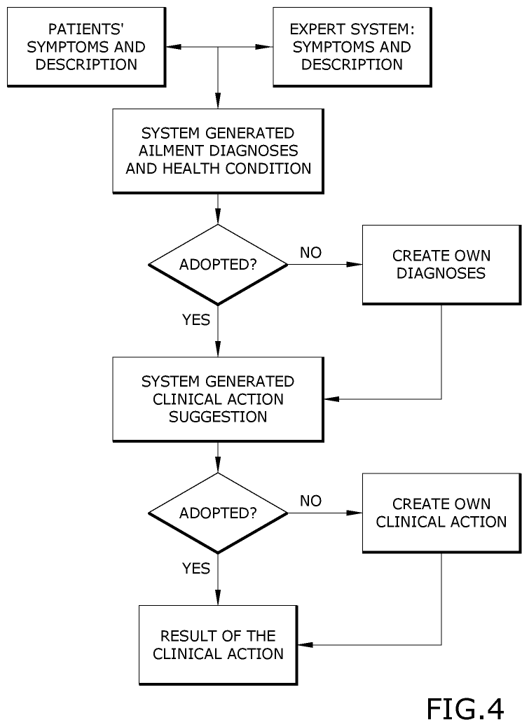

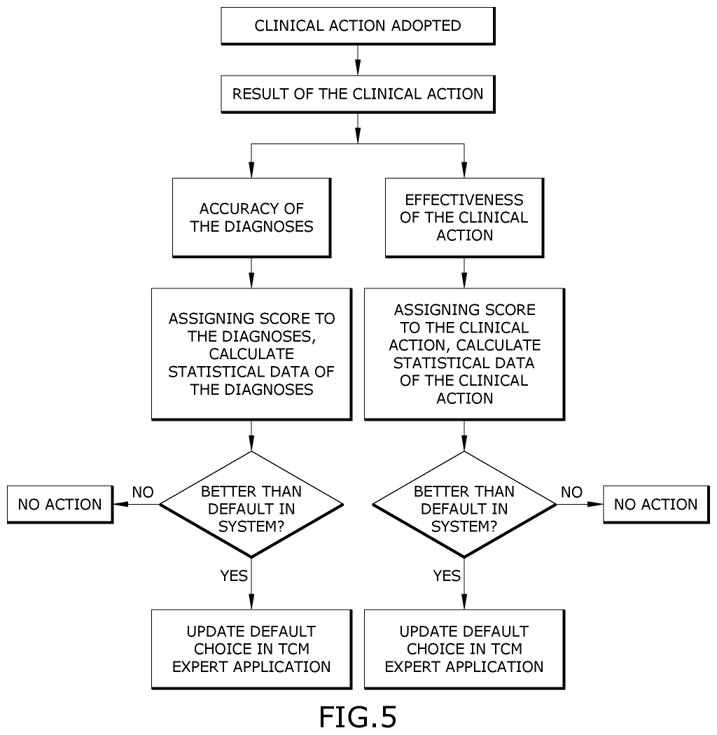

Referring now to FIGS. 1 through 5, the present invention may include a system of TCM collaboration. The system includes at least one computing device 11 having a processor and a memory. The memory includes software in the form of computing device-executable instructions that, when executed by the processor, cause the processor to implement a TCM collaboration platform 12 having, but is not limited to, a data storage and management module 30; a client-server communication module 40, a processing and data analysis module 50; and an input module 60. The TCM collaboration platform 12 couples a plurality of democratized individual client systems 10 and client systems with local centralized servers 16.

Each client system 10 has at least the processor 18 coupled to local data storage module 26 and a data input device 14. The data input devices 14, through the processor 18, may provide a data transfer and communication module 22, a local control and processing module 24, and an input module 28. Each client systems with local centralized servers 16 may have a plurality of computing devices 70 coupled to a relational database 80. The individual of computing devices 70 may include a smart phone, a tablet computer, a laptop, a desktop, and the like. The computing device 70 may execute on any suitable operating system such as IBM's zSeries/Operating System (z/OS), MS-DOS, PC-DOS, MAC-iOS, WINDOWS, UNIX, OpenVMS, ANDROID, an operating system based on LINUX, or any other appropriate operating system, including future operating systems.

In particular embodiments, the computing device 70 includes a processor, a local data storage module 76, a user interface, and a data transfer and communication module 72, a local control and processing module 74, and an input module 78. In particular embodiments, the processor includes hardware for executing instructions, such as those making up a computing device program. The memory includes main memory for storing instructions such as computing device program(s) for the processor to execute, or data for processor to operate on. The local data storage module 76 may include an HDD, a floppy disk drive, flash memory, an optical disc, a magneto-optical disc, magnetic tape, a Universal Serial Bus (USB) drive, a solid-state drive (SSD), or a combination of two or more of these. The local data storage module 76 may include removable or non-removable (or fixed) media, where appropriate. The local data storage module 76 may be internal or external to the computing device 100, where appropriate. In particular embodiments, the local data storage module 76 is non-volatile, solid-state memory.

The TCM collaboration platform 12 provides an operation system (OS) for each processor-controlled devices, modules and services, data input and request processing module, a TCM-knowledge module, an Electronic Health Record (EHR) module, a TCM expert module, a TCM Evaluation, Rating, and Modification (ERM) module, and a data management module.

The cloud-server TCM collaboration platform 12 contains the server-side data storage module 30, communication module 40, and control, processing, and data analysis module 50 as well as the input module 60.

The client system 10 contains the client-side data input module 28, data storage module 26, communication module 22, and control and processing module 24. Modules may be built on top of the Operating System (OS).

### The Data Input and Request Processing Module

The data input and request processing module may be the gateway of the system. The EHR module, the TCM-knowledge module, and the TCM Expert Module are behind the gateway to respond the request instruction. These three modules can respond to the instruction individually or via any combination among the three. The TCM expert module is coupled to a knowledge database 80. The initial content of the knowledge database 80 for the TCM Expert module may be copied from the TCM-knowledge module. The TCM ERM module may have its own database which extracts data from the EHR module. It may also have a database structure corresponding to the database 80 of the TCM expert module. The data management module has access to the database 80 of each module and controls the flow of data among the modules.

### The Request Processing Module

Data input and request processing module receives various requests from various types of users and modules through communication modules to invoke other modules inside the centralized TCM system to accomplish the request. The measurement data of pulse characteristics, color/shape/texture of the tongue, symptoms of the ailment, facial appearance, history of the ailment, and many other datapoints of the patient are collected by using four TCM methods including: Observing (wang), Listening and Smelling (these two are grouped together; wen), Asking (wen), and Touching (i.e., pulse feeling, qie). This information may be input into and stored in the EHR module. Each symptom may be input with its corresponding severity or amplitude. The information regarding the patient's medical condition is input using the TCM terms. These terms are standardized within the cloud system.

### TCM-Knowledge Module

The TCM-knowledge module encompasses various TCM theories, traditional practices, and examples. This includes Chinese medicines and their modules. The database of this module also stores holistic Chinese medicinal remedies such as food-as-medicine and other traditional do-it-yourself healing methods.

### EHR Module

The EHR module establishes, accesses and stores patient's social identification, physiological, and medical information.

### The TCM Expert Module

The TCM expert module records the information of patient's symptoms and generates diagnoses based on TCM knowledge in its database. After the TCM practitioners either adopt system-generated diagnoses or provide their own diagnoses, the TCM Expert Module generates one or more treatment instructions to each patient based on the diagnoses decided by the TCM practitioners. The primary index field of the database in this module is the ailments classified based on TCM definitions.

### TCM Evaluation, Rating, and Modification Module (ERM):

The TCM ERM module is configured to extract historical data of patients current and previous in the system. The data, instruction, and requests created at the client-side flow through the data input and request processing module to the EHR module and/or TCM-knowledge module as well as the TCM expert system module on the server side.

Depending on the request, any one or any combination of the three modules will generate feedback. When the TCM practitioners input the symptoms and other patient-related information into the TCM expert module, the TCM Expert module will generate a diagnosis. In an interactive process, the practitioners can accept the system-generated diagnoses or suggest their own. After the practitioners confirm the diagnosis, the system generates a treatment plan and provides it to the practitioners.

The TCM ERM module then processes the input and extracts the information regarding the symptoms, diagnosis, and medical treatment with regards to the ailments of the patient. The information is recorded into the database. The system generates a score to the accuracy of the diagnosis and a score to the efficacy of the treatment plan based on the improvement of the patient's health.

The comparisons of the diagnoses and medical treatment instructions among the cases stored in the system are judged by the rules and algorithm established by TCM experts based on TCM theories and the scores of the previous results.

Based on the results of the comparison, if the diagnosis and treatment method applied to the patient is better than the ones in existing theories or ones in the system, or in the situation the method has been already modified, the new treatment plan may replace the existing one in the system. The medication of the TCM method will be documented. This modification process could also be automatically executed by leveraging Machine Learning (ML) module 90. The associated data management module controls the data flow within the system. It enables the data to be transported among the modules efficiently.

### The Data Management Module

Data Management Module manages all databases in the cloud TCM system. The TCM-Knowledge module, EHR Module, the TCM Expert Module, and the TCM ERM modules each have their own relational database. The data management module controls the data flow within the system. It enables the data to be transported among the modules efficiently and securely inside the system.

The data, instruction, and requests created on the client-side flow through the data input and request processing module to the electronic health record (EHR) module and/or TCM-Knowledge module as well as the TCM Expert system module on the server-side FIG. 3.

The TCM collaboration platform 12 has different type of users, such as patients, medical authorities, and TCM practitioners, each with different access permissions. At the login stage, the system will authenticate the users and assign the corresponding access permissions based on the type of user. After the TCM practitioners input the required information, the system generates a diagnosis. The practitioners have the choice of accepting the system-generated diagnosis or submit their own. After the TCM practitioners decide to accept the suggested diagnosis or submit their own, the system suggests a treatment method. At this stage, the practitioners also have the choice of using the system-suggested medical treatment or submit their own.

The client-server communication module 40 is for accessing the TCM collaboration platform 12 over a network. The client-server communication module 40 includes hardware, software, or both providing one or more interfaces for communication (e.g., packet-based communication) between the computing device and one or more other computing devices on one or more networks. As an example, and not by way of limitation, communication interface may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 106. As an example, and not by way of limitation, the computing device 100 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks 120 may be wired or wireless. As an example, the computing device 100 may communicate with a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The computing device 100 may include any suitable communication interface 103 for any of these networks 120, where appropriate.

The TCM collaboration platform 12 may include a standalone software program, such as a social media program or an e-collaboration program. The standalone program may include a plurality of different user's that create profiles with identifying pictures, videos, personal data, and the like. Alternatively, the TCM collaboration platform 12 may include an module programming interface (API) plugin as a video commenting plugin tool that agnostically integrates a video commenting feature into already existing software platforms, web servers, and mobile based platforms. The API plugin may integrate the video commenting feature described in more detail below into existing e-learning platforms, such as but not limited to LMS and MOOCS, e-commerce, social media network platforms, news platforms, media platforms, blog platforms, product and service website platforms, and the like to enable real time video response.

A method of making the present invention may include the following. The hardware can be purchased, made from necessary components, or rented from cloud service providers. The Operating System will be bundled with the hardware or purchased separately from vendors. The software modules may be developed by software developers based on the module requirements defined in the aforementioned items and relationships. A remote/telehealth TCM practitioner visit module can be added to the system. An interface with medical institution management software can be added.

A method of using the present invention may include the following. The TCM collaboration platform 12 disclosed above may be provided. There are several types of users including general public, subscribed public users, TCM practitioners, and medical institutions.

By activating the interface to the system, public users can access part of the TCM-knowledge module. Subscribed public users can view all content the public users have access to as well as some information of their own health record. They also have limited access to some results of the TCM ERM module. The TCM practitioners can access all the areas accessible to the subscribed public users in addition to the TCM expert module, some areas of the EHR module, and more data from the TCM ERM module. Medical institutions have access to some data in the EHR module and TCM evaluation, rating, and modification module.

Therefore, this cloud based TCM platform, vis-à-vis the statistical significance of the scores of diagnostic accuracy and efficacy of treatment methods provides a foundation for standardized in TCM.

Also, the present invention can create software packages, mobile applications, and the like.

It should be understood, of course, that the foregoing relates to exemplary embodiments of the invention and that modifications may be made without departing from the spirit and scope of the invention as set forth in the following claims.

What is claimed is:

1. A system for improving Traditional Chinese Medicine (TCM) comprising:
a processor, and
a memory comprising computing device-executable instructions that, when executed by the processor, cause the processor to implement:
a communications interface for accessing a TCM collaboration platform over a network;
a user interface for displaying and interacting with the TCM collaboration platform;
a TCM-Knowledge (TCMK) module encompasses various TCM theories, traditional practices, and examples. This includes Chinese medicines and their modules.
a TCM Expert (TCME) module records the information of patient's symptoms and generates diagnoses based on TCM knowledge in its database. After the TCM practitioners either adopt system-generated diagnoses or provide their own diagnoses, the TCME Module generates one or more treatment instructions to each patient based on the diagnoses decided by the TCM practitioners. The primary index field of the database in this module is the ailments classified based on TCM definitions. TCME module retrievably storing a plurality of TCM parameters, wherein the TCMK module is configured to generate one or more of the plurality of TCM parameters upon request, wherein each TCM parameter comprises a TCM diagnosis and at least one TCM clinical action;
an Electronic Health Record (EHR) module retrievably social and medication information of each individual target; and
a TCM Evaluation, Rating, and Modification (ERM) module generates statistical evaluations of said TCM parameters and TCM practitioners,
wherein the ERM module extracts data from the EHR module to generate an efficacy score of each TCM parameter, wherein an amplitude of each efficacy score is based on improvement of the respective individual target, and wherein the efficacy score is fed back into the TCMK module for the associated TCM parameter.

2. The system of claim 1, wherein the ERM module will generate an accuracy score and statistical value for each TCM diagnosis and TCM clinical action, respectively, based on the associated efficacy score.

3. The system of claim 2, further comprising a machine learning module configured to process rules to modify the TCMK module by training the machine learning module, comprising repeatedly:
receiving an efficacy score;
accessing the TCM parameter corresponding at least in part to the efficacy score; and
identifying and extracting one or more TCM parameter stored in the TCME module based on said efficacy score.

4. The system of claim 3, wherein the machine learning module replaces the extracted TCM parameter with another TCM parameter having an efficacy score of a higher amplitude,
whereby improving the machine learning module accuracy in identifying TCM parameter deficiencies and improving the system.

5. The system of claim 4, wherein the machine learning module is configured to process rules to identifying and extracting one or more TCM diagnosis and TCM clinical action from each TCM parameter stored the TCMK module based on each respective accuracy score.

6. The system of claim 5, wherein the machine learning module replaces the extracted one or more TCM diagnosis and TCM clinical action with another one or more TCM diagnosis and TCM clinical action having an accuracy score of a higher amplitude.

7. The system of claim 6, further comprising a database management module configured to manage the data of the TCM collaboration platform to form a network-like data structure.

8. The system of claim 7, wherein the network is a cloud-based network.

9. A method for improving Traditional Chinese Medicine (TCM) using the system of claim 6, the method comprising:
inputting data of an individual target via the user interface, the data is stored in the EHR module and synchronized with the TCM collaboration platform;
using the data, requesting from the TCME module at least one recommended TCM parameter, wherein the communications interface sends the at least one recommended TCM parameter back to said user interface;
wherein a the TCM practitioner at the user interface has an option to accept or modify the at least one recommended TCM parameter as a finalized TCM parameter; and
at subsequent visit from the individual target, a result of the finalized TCM parameter are input into the TCM collaboration platform;
generating an efficacy score and statistical value for said finalized at least one TCM parameter based on the results, forming a closed loop process,
wherein a deviation of treatment from existing TCM rules is identified and evaluated by the ERM module so that if a new TCM parameter is more effective than an existing one, the recommended TCM parameter will be modified to reflect the improvement.

10. The method of claim 9, wherein the machine learning module is configured to constantly adopt the most effective TCM parameters.
